(19) United States
(12) Patent Application Publication
(10) Pub. No.: US 2026/0009093 A1
(43) Pub. Date: Jan. 8, 2026

(54) ANIMAL PATHOGEN SURVEILLANCE SYSTEM

(71) Applicant: DARO, LLC, Lincoln, NE (US)

(72) Inventors: Kristen Bernhard, Elkhorn, NE (US); Alison Neujahr, Waverly, NE (US); Josh DeMers, Howells, NE (US); Bailey Barcal, Gretna, NE (US)

(21) Appl. No.: 19/256,939

(22) Filed: Jul. 1, 2025

Related U.S. Application Data
(60) Provisional application No. 63/668,019, filed on Jul. 5, 2024.

(51) Int. Cl. C12Q 1/70; C12N 15/10; C12Q 1/6806; C12Q 1/6869; G16B 30/00; G16B 40/20; G16H 50/80

(52) U.S. Cl. CPC C12Q 1/701; C12N 15/1006; C12N 15/1096; C12Q 1/6806; C12Q 1/6869; G16B 30/00; G16B 40/20; G16H 50/80

(57) ABSTRACT

A method may include sampling feces from a floor of the facility. The method may include isolating one or more pathogens or nucleic acids from one or more pathogens from sampled feces. The method may include detecting the one or more pathogens from the sampled feces based on the nucleic acids from the one or more pathogens. The method may report a detection of the one or more pathogens.

### CROSS-REFERENCE TO RELATED APPLICATION

The present application claims the benefit under 35 U.S.C. § 119 (e) of U.S. Provisional Application Ser. No. 63/668,019, filed Jul. 5, 2024, which is incorporated herein by reference in the entirety.

### BACKGROUND

One of the most vulnerable infrastructures in society is protein food production. For example, the swine industry is susceptible to outbreaks, such pathogens as African Swine Fever virus (ASFv), Swine Influenza virus, and Porcine Reproductive and Respiratory Syndrome Virus (PRRSv). Many of these pathogens are endemic in swine facilities, resulting in lower production and animal death. In extreme years, infectious disease kills over 10% of the pigs in production in the United States. However, there are currently no pathogen surveillance systems with the necessary power and scope available to resolve these issues.

Animal testing has been used previously to determine whether specific individuals harbor specific pathogens. However, testing individual animals of a herd is time-consuming, and expensive. Further, while individual testing assists producers in identifying animals harboring pathogens, the testing does not effectively predict pathogen outbreaks in time for producers to utilize disease mitigation efforts.

Therefore, it is advantageous to provide a method that overcomes the challenges described above.

### SUMMARY

A method for surveilling a population of animals within a facility for one or more pathogens is disclosed. In embodiments, the method includes sampling feces from a floor of a facility. In embodiments, the method includes isolating the one or more pathogens or nucleic acids from the one or more pathogens from a sampled feces. In embodiments, the method includes detecting the one or more pathogens from the sampled feces based on the nucleic acids from the one or more pathogens from the sampled feces. In embodiments, the method includes reporting a detection of the one or more pathogens.

A kit for surveilling a population of animals within a facility for one or more pathogens is disclosed. In embodiments, the kit includes a shoe cover configured to absorb feces. In embodiments, the kit includes an immersion container configured to receive the shoe cover and a diluent, wherein the shoe cover has absorbed feces, wherein the immersion container is configured to contain a slurry formed from an addition of the diluent to the shoe cover. In embodiments, the kit includes a collection container configured to receive the slurry from the immersion container.

It is to be understood that both the foregoing general description and the following detailed description are exemplary and explanatory only and are not necessarily restrictive of the present disclosure. The accompanying drawings, which are incorporated in and constitute a part of the specification, illustrate subject matter of the disclosure. Together, the descriptions and the drawings serve to explain the principles of the disclosure.

### BRIEF DESCRIPTION OF THE DRAWINGS

The numerous advantages of the disclosure may be better understood by those skilled in the art by reference to the accompanying figures.

FIG. 1 illustrates a process flow diagram depicting a method for surveilling a population of animals within a facility for one or more pathogens.

FIG. 2 illustrates a kit for the collection of a biological sample, in accordance with one or more embodiments of the disclosure.

FIG. 3 illustrates an amplification plot for biological samples tested for porcine reproductive and respiratory syndrome virus (PRRSv), in accordance with one or more embodiments of the disclosure.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

Methods for pathogen detection and forecasting the progression of disease in an animal herd are provided. The methods rely on detecting and/or determining the level of at least one biomarker (e.g., a nucleic acid sequence or Ct value) or combinations of biomarkers in a collected and/or pooled sample from the feces of a population of animals. The feces may be collected from the floor of a facility, such as a livestock housing area or confinement area. The methods may include one or more steps of collecting and/or pooling biological samples (e.g., feces) from a floor of the facility, isolating nucleic acids from pathogens collected from the collected and/or pooled feces, detecting one or more pathogens from the isolated nucleic acids, and reporting a detection of the one or more pathogens.

The methods provide an accurate reflection of pathogen load across a population of animals, such as a swine herd. The methods are sensitive, with detection rates reaching one viral shedding animal per 1000 or more healthy or non-shedding animals. Once test results are collected, the data may then be sent to producers as reports that may include findings of pathogen detection and/or forecasts of pathogen spread within the animal herd.

The methods of the present disclosure are of particular advantage over other pathogen detection methods for animal herds. For example, while human wastewater has been used previously for detection of community viral load, the use of manure pits for pathogen detection in swine herds would not be accurate due to the long periods of time that the manure and feces in the pits are stagnant. The methods of this disclosure provide whole herd pathogen surveillance at a relatively low cost per animal and does not require direct interaction with the animals.

In embodiments, the methods include the collection of a biological sample from a plurality of animals (e.g., domestic animals or nondomestic animals). For example, the methods may include collecting biological samples from mammals, such as mammals used as livestock, including, but not limited to, pigs, cattle, sheep, goats, and horses. For instance, the methods may include collecting swine biological samples from the floor and/or other surfaces of a pig confinement room. The methods may include collecting biological samples from other animals including, but not limited to, birds (e.g., chickens and turkeys), reptiles, and fish.

In embodiments, the methods include collecting the biological material from the floor and/or other surfaces of the facility. Collecting the biological material may include contacting the floor and/or other surfaces with an absorbent article that absorbs the biological material. The absorbent article may include any type of absorbing item or material including, but not limited to, towels, rags, pads, and garments. For example, the absorbent article may include footwear, such as a shoe cover. For instance, the footwear may include a shoe cover having an absorbing sole surface.

Biological material may be collected from multiple animals, depending on the number of herd animals and access of the herd animals to the facility. However, portions of the defecations may remain temporarily on the facility floor, creating a fresh fecal mix as the animals walk through the facility. For collection of the biological sample, an operator may walk on the facility floor with absorbent shoe covers, which may absorb portions of the fecal mix, potentially collecting a biological sample of every animal, or nearly every animal, which has defecated on the facility floor. The collection may stop when the shoe covers are saturated. For example, the operator may wear multiple layers of shoe covers, removing each layer one at a time as they become saturated. Using this collection scheme, an operator may collect pooled biological samples from more than ten animals at a time, from more than 100 animals at a time, or from more than 1000 animals at a time (e.g., assuming that many or most animals that have been placed in a pen will leave behind fecal material). For example, an operator may collect and pool samples from 1100 animals by sampling six pens in a single facility using one or more absorbent articles that are then collected to create a pooled sample.

The amount of biological material collected by each absorbent article may be between one gram and 100 grams. For example, approximately 10 grams of feces may be collected from a shoe cover. When collecting the biological material, it may be beneficial to remove fecal chunks, if any, from the absorbent article, as these chunks may include biological material from a single individual or a few individuals, which may skew results when collecting biological material from a larger herd of animals.

In embodiments, the absorbent articles (e.g., shoe covers) are placed into a pouch, bag, or other type of container (e.g., immersion container) and immersed with a diluent (e.g., water or a water-containing solution). The immersion facilitates the extraction of the biological sample from the absorbent article. For example, one or more absorbent articles (e.g., shoe covers) saturated or partially saturated with biological samples may be placed in a bag (e.g., a resealable plastic bag). Water or other extraction liquid may then be added to the bag. The amount of liquid added to the bag may vary as appropriate. For example, each absorbent article may be extracted with approximately 200 ml of fluid. For instance, ten absorbent articles may be placed in a single bag with approximately two liters of fluid (e.g., an approximately 1:20 dilution of estimated grams of biological sample to milliliter (ml) of fluid). Air may be purged from the bag after the addition of the absorbent materials and the fluid.

In embodiments, the bag (e.g., immersion container) containing the absorbent articles and the fluid is mixed and/or shaken. For example, the bag may be shaken vigorously for approximately 30 seconds. Once thoroughly shaken and mixed, the bag is opened, and a slurry containing the biological sample may be poured and/or decanted into a sample tube, such as a 15 ml tube. In some cases, the slurry may first be poured into a cup (e.g., a collection cup or paper cup) or other wider-mouthed container before being poured into the sample tube. The sample tube may then be capped and labeled.

In embodiments, the biological material (e.g., stored in a sample tube) is stored at less than room temperature before being tested for one or more pathogens. For example, the biological material may be refrigerated to approximately 4° C. and stored for up to two weeks. In another example, the biological material may be frozen at approximately −20° C. for up to two months. In another example, the biological material may be frozen to approximately −80° C. and stored indefinitely. In another example, the biological material may be stored along with frozen cold packs for up to 72 hours for shipping.

In embodiments, the pathogens and/or nucleic acids from the pathogens are extracted from the slurry. The extraction of the pathogens from the slurry requires care to remove and/or inhibit inhibitors of polymerases for downstream applications. For example, several substances in corn (e.g., acidic plant polysaccharides, humic acids, and polyphenols) are known to inhibit polymerases and PCR reactions. Because of this, livestock animals that have corn-specific diets, such as pigs, may produce biological samples that inhibit PCR amplification.

In embodiments, extraction of pathogen and/or nucleic acids from the slurry includes a step of diluting the slurry. For example, a technician may receive a slurry containing the biological sample (e.g., a 1:20 dilution of the slurry) and dilute the slurry further before continuing on with the extraction steps. Diluents used for diluting the slurry may include, but not be limited to, water, or a dilution buffer (e.g., Tris-buffered saline or Phosphate buffered saline). The resultant diluted slurry may include a 1:5 dilution of the slurry, a 1:10 dilution of the slurry, a 1:20 dilution of the slurry, a 1:30 dilution of the slurry, a 1:40 dilution of the slurry, a 1:50 dilution of the slurry, or approximate dilutions. Because the slurry is considered a dilution of the original biological sample (e.g., 1:20 dilution), the above dilutions may also be considered as a 1:100 dilution of the biological sample, a 1:200 dilution of the biological sample, a 1:400 dilution of the biological sample, a 1:600 dilution of the biological sample, a 1:800 dilution of the biological sample, a 1:1000 dilution of the biological sample, a 1:2000 dilution, a 1:4000 dilution, a 1:8000 dilution, or approximate dilutions, respectively. The increased dilutions may assist in preventing the inhibition of downstream amplification and detection methods by compounds residing in the biological sample.

In embodiments, the extraction includes adding trapping particles that trap pathogens (e.g., viruses and/or microbes) and/or pathogen nucleic acids (e.g., pathogen components). For example, the extraction may include the addition of beads and/or other particle-based formulations that bind to the pathogens or pathogen components. Several types of trapping particles for extracting pathogens and pathogen components waste slurries are commercially available. For example, the trapping particles may include affinity-capture magnetic hydrogel particles.

In use, the trapping particles may be added directly to the slurry or to a diluted slurry. Multiple types of trapping particles may be used per sample, which may assist in increasing the amount and/or diversity of pathogenic material. Once the trapping particles have been added to the slurry, the slurry is mixed (e.g., via inversion). The slurry may further be incubated (e.g., at room temperature). For example, the slurry may be incubated at room temperature for approximately 10 minutes. A supernatant may then be separated from the trapping particle/slurry mix via settling, centrifugation, filtering, and/or magnets, depending on the type of trapping particles used. While the pathogens and/or pathogen components remaining on the trapping particles may be used directly for downstream applications, the trapping particles may be washed one or more times if needed. For example, after removing the initial supernatant, a small amount (e.g., 1 ml) of water or buffer may be added to the trapping particles. The trapping particles may then be resuspended, and the wash then removed as described above. The water used in resuspending the trapping particles may be highly purified (e.g., molecular grade) water. The water and/or buffer may also be used to transfer the trapping particles to a new tube (e.g., a 1.5 ml tube).

In embodiments, the extraction includes a resuspension of the trapping particles with a lysis buffer. The lysis buffer may cause one or more of a lysis of pathogens (e.g., microbes and viruses) bound to the trapping particles, a denaturing of pathogens (e.g., viruses) bound to the trapping particles, and/or a release of pathogenic components (e.g., nucleic acids) from the trapping particles. The amount of lysis buffer used may depend on the amount of trapping particles and/or original amount of biological sample. For example, 500 microliters (μl) of lysis buffer may be added to the trapping particles.

Once resuspended, the slurry formed from the trapping particles and the lysis buffer may be mixed, inverted, and/or heated. For example, the trapping particles may be heated at 90° C. or greater for more than one minute. In another example, the trapping particles may be heated at 95° C. for 10 minutes. The heating both denatures proteins and nucleic acids and facilitates the removal of the pathogen and pathogen components from the trapping particles. After the heating step, the trapping particles are pelleted via methods mentioned herein. The supernatant is then removed and placed in a new tube as a lysate.

In embodiments, the extraction includes an addition of nucleic acid binding substrate (e.g., binding beads) to the lysate (e.g., a supernatant containing the nucleic acids from the one or more pathogens), mixing the lysate/bead mixture, and incubating the lysate/bead mixture for an incubation period. The binding beads may be configured to bind pathogenic nucleic acids, such as viral genomic RNA or DNA in the lysate, enabling the nucleic acids to be further purified and concentrated. Beads that bind nucleic acids are well-known and are commercially available.

In embodiments, the extraction includes a step of adding a component that degrades and/or denatures protein. For example, the extraction may include the addition of proteinase K, which digests proteins from the biological sample. Proteinase K may be added in one or more steps within the extraction method. For instance, the proteinase K may be added when the lysate is incubated with the binding beads. Once added, the slurry/lysate may be incubated to a temperature wherein proteinase K is active. (e.g., 37° C. to 70° C.). For example, the slurry/lysate containing proteinase K may be incubated at 65° C. for approximately 5 minutes, approximately 10 minutes, or approximately 15 minutes or more.

In embodiments, the extraction includes a step of adding an internal RNA positive control. The internal RNA positive control provides a reference for determining whether the extraction steps and downstream amplification/sequencing steps have been correctly performed. For example, the internal RNA positive control may assist in determining if inhibitors from the biological sample are inhibiting downstream amplification and/or sequencing method steps. An example of an RNA positive control is the VetMAX™ Xeno™ Internal Positive Control RNA available via the ThermoFisher company. Other internal RNA positive controls may include, but not be limited to, viral genomes or RNAs sourced from commercially available vaccine preparations.

After the incubation period, the binding beads may be washed and resuspended. The binding beads may then be collected via centrifugation, settling, magnetism, or filtering. The supernatant may then be removed, leaving the binding beads along with the bound pathogenic nucleic acids. The binding beads may then be washed one or more times, with a resuspension and collection step being performed at each wash. For example, the binding beads may be washed with one or more washes of an aqueous buffer and/or an ethanol-containing wash (e.g., 80% ethanol).

After the washing steps, the nucleic acids may be eluted from the binding beads by resuspending the binding beads via an addition of an elution buffer (e.g., into an eluted sample). The elution may include incubating the binding beads for an extended time (e.g., 10 minutes). The elution may further include incubating the binding beads at an elevated temperature. For example, the binding beads may be incubated at 65° C. After incubation of the binding beads in the elution buffer, the beads are collected, and the supernatant containing the eluted nucleic acids is transferred to a new tube (e.g., as a nucleic acid sample) and may be stored at −20° C. or colder temperatures.

In embodiments, the method includes detecting one or more pathogenic nucleic acids from the nucleic acid sample. The detection of one or more pathogenic nucleic acids from the nucleic acid sample may include an amplification step and/or a sequencing step. For example, for a pathogen possessing an RNA genome or an mRNA, RNA may be reverse-transcribed into cDNA, and the cDNA may then be amplified as part of a qualitative PCR (qPCR) method for detecting a specific nucleic acid sequence of the pathogen genome. In another example, for a pathogen possessing a DNA genomic, qPCR may be performed without a reverse-transcription step. In embodiments, detecting one or more pathogenic nucleic acids from the nucleic acid sample includes performing nanopore sequencing, where nucleic acids are passed through a synthetic nanopore that detects individual bases and/or other biomolecules based upon changes in electrical conductivity. Other types of nucleic acid sequencing, next-generation sequencing (NGS), and nucleic acid detection protocols may also be utilized. For example, nucleic acid sequencing protocols developed and/or shared by the ARTIC Network may be utilized to detect and/or sequence a portion of, or the entirety of, pathogens such as PRRSv. In another example, shotgun metagenomics may be used to analyze the genetic material of all organisms within a sample. In another example, focused nucleic acid sequencing (e.g., 16S rRNA sequencing) may be used to identify bacterial pathogens in a sample.

The pathogens detected by the method may include a single pathogen or multiple pathogens. For example, using PCR-based protocols, the method may be used to detect a single pathogen. In another example, using multiplex PCR-based protocols, the method may be used to detect two or more pathogens, four or more pathogens, eight or more pathogens, 16 or more pathogens, or 32 or more pathogens. In another example, using NGS sequencing, where sequencing is not restricted to specific primer pairing, the method may be used for the detection of any pathogen chosen to be under surveillance.

The pathogens under surveillance by the method may include any pathogen for any animal as described herein. For example, the pathogens under surveillance may include swine pathogens including, but not limited to, swine influenza virus (e.g., influenza A), porcine reproductive and respiratory syndrome virus (PRRSv), porcine epidemic diarrhea virus (PEDv), deltacoronavirus (e.g., human porcine deltacoronavirus or HuPDCoV), transmissible gastroenteritis (TGEv) and Mycoplasma hyopneumoniae. For example, the method may include amplification and detection of at least a portion of PRRSv. For instance, the method may include the amplification and detection of a region of PRRSv that includes at least a portion of one or more of the ORF4 (open reading frame four) gene, the ORF5 gene (open reading frame five), or the ORF6 gene (open reading frame six). In another example, the method may include a multiplex amplification method that includes the ability to amplify portions of swine influenza virus, PRRSv, PEDv, deltacoronavirus, TGEv, and Mycoplasma hyopneumoniae within the same multiplex reaction. In another example, the method may include a multiplex amplification method that includes the ability to amplify portions of five of the six pathogens of swine influenza virus, PRRSv, PEDv, deltacoronavirus, TGEv, and Mycoplasma hyopneumoniae within the same multiplex reaction.

In embodiments, the method includes reverse transcribing nucleic acids from the nucleic acid sample with an inhibitor-resistant reverse-transcriptase. For example, the reverse-transcriptase used for reverse-transcribing genomic RNA from the nucleic acid sample may be one that is resistant to the enzyme inhibitors often found in swine fecal samples, particularly swine fecal samples from pigs fed a corn diet. For instance, the method may include using the reverse transcriptase SuperScript™ IV produced by the Invitrogen company.

For methods that utilize an amplification step, the amplified material may be purified before the sequencing and/or detection step. Methods for purifying the amplified material may include, but not be limited to, phenol extraction, ethanol precipitation, column purification, and bead-based purification.

In embodiments, the method includes making a quality measurement of the purified or semi-purified nucleic acids (e.g., before or after transcription and/or amplification). For example, after the nucleic acids have been amplified, a portion of the amplified nucleic acids may be analyzed through electrophoresis. For instance, the amplified nucleic acids may be analyzed via a TapeStation™ system produced by the Agilent company.

In embodiments, the results of the amplification and sequencing are analyzed. For example, data from the amplification and/or sequencing results are input into a computer, where bioinformatic software identifies the amplification and/or sequencing result as a detection of one or more pathogens. Software for qualitatively or quantitatively determining the presence of a pathogen is commercially available. For example, machine learning (ML) or artificial intelligence (AI) platforms may be used to create regional disease load forecast information that can be displayed on regional and/or national dashboards, as well as localized barn and enterprise monitors. For example, reports based on these analyses may be displayed on barn monitors and producer alert systems.

In embodiments, the results of the tests are reported, such as to workers, managers, owners, and other stakeholders of the herd. For example, the results may be configured as computer files (e.g., spreadsheet files or PDFs) that are sent to a computing device (e.g., computer, laptop, smartphone, tablet) of the manager or owner.

In embodiments, the analysis from the amplification and sequencing are further analyzed for disease forecasting. For example, pathogen-associated data may be entered into models, such as AI/ML models, which can be used to predict how a pathogen may spread (e.g., through a facility or regionally). For example, machine learning platforms may be used to create regional disease load forecast information that can be displayed on regional and/or national dashboards, as well as localized barn and enterprise monitors. For example, reports based on these analyses may be displayed on barn monitors and producer alert systems. Through early detection via fecal monitoring and disease forecasting, the method described herein may provide livestock producers with accurate and early detection of swine pathogens across their herds and may be able to make quick and accurate action related to herd health and productivity while lessening the possibility of further pathogen spread. For example, the AI/ML models may, based at least on a detection of one or more pathogens, infer a forecast of a disease state for the population of animals. Examples of platforms for reporting results from the surveillance testing include, but are not limited to, the UC Davis platform, Disease Bioportal, and the barn alarm platform, Distynct.

In embodiments, the methods of the current application can detect one or more pathogens in a population of animals at least one day before other surveillance methods, such as oral fluid surveillance methods. For example, the methods of the current application can detect one or more pathogens in a population of animals on average at least one day before other surveillance methods, such as oral fluid surveillance methods. Oral fluid surveillance methods may include using cotton ropes that are suspended at shoulder height in a pen containing one or more pigs, typically for 20 to 30 minutes. The pigs chew on the ropes, which absorb saliva and oral mucosa. These ropes are then processed for pathogen detection.

In embodiments, one or more pathogens, such as pathogens of swine (e.g., swine influenza virus, PRRSv, PEDv, deltacoronavirus, TGEv, and Mycoplasma hyopneumoniae) are detected by the method of the current application at least one day before the oral surveillance (e.g., oral fluids) method. For example, the method of the current application may detect one or more of the above pathogens two or more days before detection by the oral surveillance method. In another example, the method of the current application may detect one or more of the above pathogens three or more days before detection by the oral surveillance method. In another example, the method of the current application may detect one or more of the above pathogens four or more days before detection by the oral surveillance method. In another example, the method of the current application may detect one or more of the above pathogens at an average of at least 3.92 days before detection by the oral surveillance method.

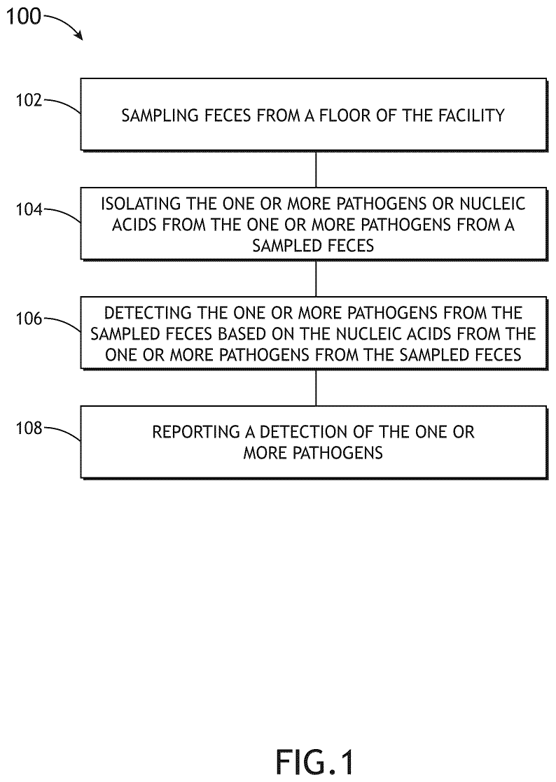

FIG. 1 illustrates a process flow diagram depicting steps in a method for surveilling a population of animals within a facility for one or more pathogens. For example, the method may be used for the detection and reporting of pathogens in a livestock facility, (e.g., a hog confinement facility) as described herein.

In embodiments, the method 100 includes a step 102 of sampling feces from a floor of the facility. Sampling feces from the floor of the facility may include walking along the floor within the facility with footwear (e.g., shoe covers), wherein the footwear comprises an absorbent cover. Sampling feces from the floor of the facility may further include absorbing the feces onto the absorbent cover, removing the footwear, and/or transferring the feces from the absorbent cover to a sample container, such as a 15 ml tube.

Transferring the feces from the absorbent cover to the sample container may include one or more of placing the absorbent cover in an immersion container, wherein the footwear comprises a shoe cover, adding a diluent to the immersion container, wherein adding the diluent transfers the feces from the absorbent cover and produces a slurry, and transferring the slurry from the immersion container to the sample container.

In embodiments, the method 100 includes a step 104 of isolating the one or more pathogens or nucleic acids from the one or more pathogens from a sampled feces. For example, the step 104 may include one of more of adding trapping particles to the slurry, wherein the trapping particles bind the one or more pathogens or the nucleic acids from the one or more pathogens, washing the trapping particles, adding a lysis buffer; wherein the lysis buffer causes a lysis of pathogens bound by the trapping particles, heating the lysis buffer and the trapping particles for at 90° C. or greater for more than one minute, removing a supernatant from the trapping particles, wherein the supernatant contains the nucleic acids from the one or more pathogens, adding nucleic acid binding substrate to the supernatant wherein the supernatant contains the nucleic acids from the one or more pathogens, washing the nucleic acid binding substrate, and eluting the nucleic acids from the nucleic acid binding substrate into an elution sample. In another example, proteinase K may be added before washing the nucleic acid binding substrate.

In embodiments, the method 100 includes a step 106 of detecting the one or more pathogens from the sampled feces based on the nucleic acids from the one or more pathogens from the sampled feces. For example, the step 106 may include one or more of obtaining a portion of the elution sample, reverse transcribing RNA from the portion of the elution sample, producing complementary cDNA, amplifying a region of a genome or mRNA of the one or more pathogens from the complementary cDNA, producing an amplified product, and detecting the amplified product. The amplified products may include a portion of one or more of PRRSv, PDCOV, PEDv, TGEV, or Mycoplasma hyopneumoniae. In another example, before washing the nucleic binding substrate, a control nucleic acid (e.g., “Xeno”) is added, which is then amplified and detected as a positive control. Before reverse transcribing the RNA, the elution sample may be treated with a DNase, such the ezDNase™ enzyme sold by Thermo Fisher Scientific.

In embodiments, the method 100 includes a step 108 of reporting a detection of the one or more pathogens. The reporting may be local (e.g., to a producer, manager, or worker), or nonlocally to a reporting institution or forecasting website. For example, reporting a detection may include providing a forecast of a disease state for a population of animals for one or more of the one or more pathogens. In another example, reporting a detection may include obtaining a trained artificial intelligence (AI) and/or machine learning (ML) model, and, based at least on the detecting of the one or more pathogens from the sampled feces and the trained AI and/or ML model, inferring a forecast of a disease state for the population of animals.

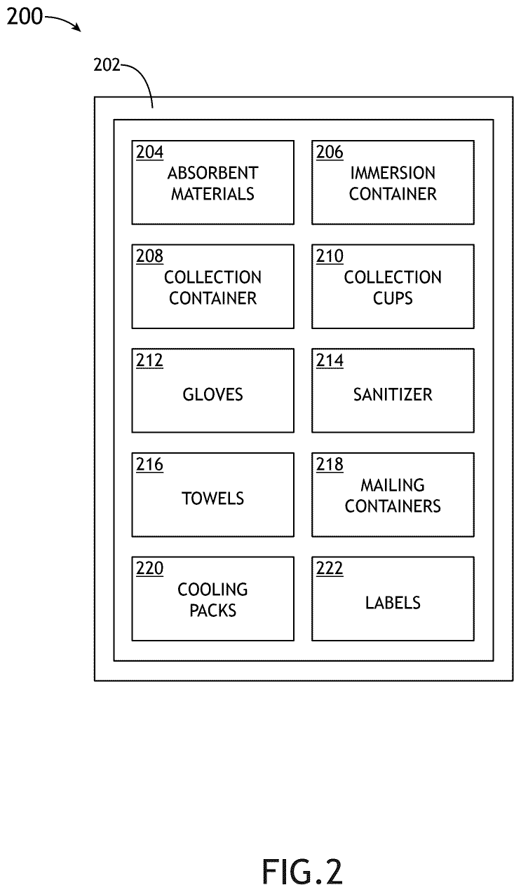

FIG. 2 illustrates a kit 200 for the collection of a biological sample, in accordance with one or more embodiments of the disclosure. For example, the kit 200 may include the materials necessary for collecting the biological material from the floor of a facility to pouring the slurry containing the biological material into a collection container that is sent to a testing center.

In embodiments, the kit 200 includes a container 202 (e.g., a box, bag, or pouch) for containing the components of the kit 200. The container may be insulated. The kit 200 may further include one or more of absorbent materials 204 (e.g., shoe covers), pouches (e.g., immersion container 206, such as resealable bags), collection containers 208 (e.g., 15 ml plastic tubes), and collection cups 210 for transferring the slurry from the immersion container 206 to the collection container 208. The kit 200 may also include one or more of gloves 212, sanitizer 214, and towels 216. The kit 200 may also include a mailing container 218 for sending the collection containers 208 to the testing center, one or more cooling packs 220 for keeping the slurry cold during transport, and/or labels 222 for labeling the collection containers 208 and or mailing container 218.

Non-limiting examples of the present disclosure will now be described in the following numbered clauses:
Clause 1: A method for surveilling a population of animals within a facility for one or more pathogens comprising: sampling feces from a floor of the facility; isolating the one or more pathogens or nucleic acids from the one or more pathogens from a sampled feces; detecting the one or more pathogens from the sampled feces based on the nucleic acids from the one or more pathogens from the sampled feces; and reporting a detection of the one or more pathogens.
Clause 2: The method of clause 1, wherein sampling feces from the floor of the facility comprises: walking along the floor within the facility with footwear, wherein the footwear comprises an absorbent cover; absorbing feces from the floor onto the absorbent cover; removing the footwear; and transferring the feces from the absorbent cover to a sample container.
Clause 3: The method of clause 2, wherein transferring the feces from the absorbent cover to the sample container comprises: placing the absorbent cover in an immersion container, wherein the footwear comprises a shoe cover; adding a diluent to the immersion container, wherein adding the diluent transfers the feces from the absorbent cover and produces a slurry produces a slurry; and transferring the slurry from the immersion container to the sample container.
Clause 4: The method of clause 3, wherein isolating the one or more pathogens from the sampled feces comprises: adding trapping particles to the slurry, wherein the trapping particles bind the one or more pathogens or the nucleic acids from the one or more pathogens; washing the trapping particles; adding a lysis buffer; wherein the lysis buffer causes a lysis of pathogens bound by the trapping particles; heating the lysis buffer and the trapping particles for at 90° C. or greater for more than one minute; removing a supernatant from the trapping particles, wherein the supernatant contains the nucleic acids from the one or more pathogens; adding nucleic acid binding substrate to the supernatant wherein the supernatant contains the nucleic acids from the one or more pathogens; washing the nucleic acid binding substrate; and eluting the nucleic acids from the nucleic acid binding substrate into an elution sample.
Clause 5: The method of clause 4, further comprising before washing the nucleic acid binding substrate, adding proteinase K.
Clause 6: The method of clause 4, wherein detecting the one or more pathogens from the sampled feces comprises: obtaining a portion of the elution sample; reverse transcribing RNA from the portion of the elution sample, producing complementary cDNA; amplifying a region of a genome or mRNA of the one or more pathogens from the complementary cDNA, producing an amplified product; and detecting the amplified product.
Clause 7: The method of clause 6, wherein the amplified product comprises a portion of a porcine reproductive and respiratory syndrome virus (PRRSv) genome.
Clause 8: The method of clause 7, wherein the amplified product comprises open reading frame five (ORF5) of the PRRSv genome.
Clause 9: The method of clause 7, wherein the method detects PRRSv in the population of animals four days before a detection by an oral fluids detection protocol.
Clause 10: The method of clause 6, wherein the slurry comprises feces from more than 100 animals of the population of animals, wherein the method is configured to detect a single positive-testing animal within the slurry.
Clause 11: The method of clause 6, wherein the amplified product comprises a portion of swine influenza virus.
Clause 12: The method of clause 6, wherein the amplified product comprises a portion of porcine deltacoronavirus (PDCoV) genome.
Clause 13: The method of clause 6, wherein the amplified product comprises a portion of porcine epidemic diarrhea Virus (PEDv) genome.
Clause 14: The method of clause 6, wherein the amplified product comprises a portion of transmissible gastroenteritis virus (TGEV) genome.
Clause 15: The method of clause 4, wherein detecting the one or more pathogens from the sampled feces comprises: obtaining a portion of the elution sample; amplifying a region of a genome or mRNA of the one or more pathogens from the elution sample, producing an amplified product; and detecting the amplified product.
Clause 16: The method of clause 4, further comprising before washing the nucleic acid binding substrate, adding a control nucleic acid, wherein the control nucleic acid is eluted in the elution sample, amplified, and detected.
Clause 17: The method of clause 4, wherein one or more of the nucleic acids from the one or more pathogens are detected via nanopore sequencing.
Clause 18: The method of clause 1, wherein reporting the detection of the one or more pathogens comprises: providing a forecast of a disease state for a population of animals for one or more of the one or more pathogens.
Clause 19: The method of clause 1, wherein reporting the detection of the one or more pathogens comprises: obtaining a trained artificial intelligence (AI) and/or machine learning (ML) model; and based at least on the detecting of the one or more pathogens from the sampled feces and the trained AI and/or ML model, inferring a forecast of a disease state for the population of animals.
Clause 20: A kit for surveilling a population of animals within a facility for one or more pathogens comprising: a shoe cover configured to absorb feces; an immersion container configured to receive the shoe cover and a diluent, wherein the shoe cover has absorbed feces, wherein the immersion container is configured to contain a slurry formed from an addition of the diluent to the shoe cover; and a collection container configured to receive the slurry from the immersion container.

The following examples are meant only to be illustrative and are not meant as limitations on the scope of the invention or of the appended claims.

### EXAMPLES

### Example 1: Collection Protocol

A protocol for collecting biological material from a floor of a swine confinement facility was performed. The protocol includes transporting a fecal sampling kit to the swine confinement facility. The fecal sampling kit included a set of shoe covers (e.g., five shoe covers for each shoe or boot), a resealable bag, a collection cup (e.g., a paper cup), and a 15 ml collection container.

The collection protocol included placing the multiple shoe covers on each foot (e.g., five per shoe), and walking the pens to be tested, making sure to walk through all dunging areas. It is noted to make sure to collect only “smears” of feces, not “chunks”. Any chunks of feces that attach to the shoe covers will need to be scraped off. Once the shoe cover (e.g., the outside most shoe cover), becomes saturated, the shoe cover is removed and placed into the resealable bag (e.g., a two-gallon resealable bag).

When all shoe covers to be used have been placed into the resealable bag, two liters of water are added to the bag. Air is expelled from the bag, and the bag is sealed tightly. The bag is then shaken for approximately 30 seconds. After shaking, the bag is opened, and a portion of the slurry is poured into the collection cup. Approximately 10 ml of slurry is then poured from the collection cup and is poured into the collection container and capped. Data regarding the collection is then written onto the collection container and/or an input form. The collection container containing the slurry was then placed into a refrigerator at 4° C. From this collection protocol, approximately 10 grams of feces were recovered from each shoe cover (e.g., 100 grams total). With the addition of the two liters of water, the approximate dilution of the feces was estimated to be 1:20, as feces and water have similar densities.

### Example 2: Enrichment of Pathogen and Pathogen Components from Slurry

Dilutions of the slurry collected in the protocol in Example 1 were made with water. For example, a 1:10 dilution (1:200 total) and a 1:30 dilution (1:600 total) were made. 100 microliters of nanotrap enhancement reagent (Ceres Nanotrap Enrichment Reagent 1 ER1 (Cat #10111)) was added to each diluted sample and inverted two times to mix. 300 microliters of nanotrap microbiome particles (Ceres Nanotrap Microbiome A Particles (Cat #44202)), were then added to each diluted sample and inverted five times to mix. The samples were then incubated at room temperature for ten minutes, inverting every five minutes.

After the room temperature incubation, the tubes containing the diluted samples were placed on a track for five minutes to separate the nanotrap particles from the rest of the slurry. Using a serological pipette, the supernatant (not containing the nanotrap particles), was removed via a pipette and discarded (e.g., in bleach solution). The nanotrap particles were then resuspended in one ml of molecular biology-grade water, making sure to pipette the water onto the tube walls. The resuspended nanotrap particles were then transferred to a new 1.5 ml microcentrifuge tube and placed onto a magnetic rack.

After two minutes, the supernatant was removed and discarded without disrupting the nanotrap pellet. The nanotrap pellet was then resuspended in 500 microliters of MagMax™ Lysis Solution obtained from the Applied Biosystems™ MagMAX™ Wastewater Ultra Nucleic Acid Isolation Kit (Cat #A52606). The tube was then placed on a heat block at 95° C. for 10 minutes.

While the sample were incubating an extraction buffer was prepared. First, an RNA/proteinase K master mix was created using ten microliters of a proteinase K solution and two microliters of an RNA control (e.g., a Xeno RNA control) for each sample. Twelve microliters of master mix were added to each transfer tube. Second, a bead master mix was created using 530 microliters of binding solution 20 microliters of binding beads (e.g., from the Applied Biosystems™ MagMAX™ Wastewater Ultra Nucleic Acid Isolation Kit). The beads were vortexed before use. 550 microliters of the bead master mix were then added to each transfer tube.

After the ten-minute incubation period, the heated samples were centrifuged briefly and placed on a magnetic rack for two minutes. 400 microliters of supernatant from the sample tubes were then added to the prepared transfer tubes. The proteinase K-containing transfer tubes were then incubated at 65° C. for 10 minutes, then centrifuged briefly and placed on a magnetic rack for two minutes. The supernatant was then removed and discarded. The beads were then washed sequentially with one ml of MagMAX™ Wash Buffer, one ml of 80% ethanol, and 500 microliters of 80% ethanol, with the tube placed on a magnetic rack for two minutes after each wash.

After the 500-microliter wash, the transfer tubes were centrifuged briefly, and the remaining 80% ethanol was removed with via a micropipette. The transfer tubes were then left to dry to ambient air for a minute. The beads were then resuspended in 60 microliters of elution solution and incubated at 65° C. for 10 minutes. Afterward, the transfer tubes were centrifuged briefly, and the supernatant containing the pathogenic nucleic acid was transferred to a new tube and stored at −20° C.

### Example 3: Detection of Pathogenic Nucleic Acids

A portion of the eluted material in Example 2 was tested for pathogenic nucleic acids, specifically for a portion of the ORF5 gene of the PRRSv (e.g., a target sequence). RNA from the eluted material was first transcribed via a reverse transcriptase reaction (Invitrogen™ SuperScript™ IV First-Strand Synthesis System, Catalog No. 18-091-050, sold by Fisher Scientific).

Before reverse transcription was performed, the sample genomic DNA within the eluted sample was digested (e.g., an option step). For this, digestions were set up, as shown in Table 1 and placed on ice.

TABLE 1

| Component | Volume |
|---|---|
| 10X ezDNAse Buffer | 1 ul |
| EzDNAse Enzyme | 1 ul |
| Template RNA (1 pg-2.5 ug total RNA) | Up to 8 ul |
| Total | 10 ul |

After mixing and brief centrifugation, the reactions were incubated at 37° C. for 2 minutes, then incubated with an inactivation step at 55° C. for 5 minutes. In an optional step, 1 ul of 10 mM DDT can be added as the reaction is heating to 55° C. After the inactivation step, the reaction tubes are gently centrifuged and placed on ice, and the RNA (e.g., template RNA) is added to an annealing mix, as shown in Table 2.

TABLE 2

| Component | Volume |
|---|---|
| 2 uM gene-specific Primer (ORF5 primer) | 1 ul |
| or random hexamers | |
| 10 mM dNTP Mix | 1 ul |
| Template RNA (10 ng total) or RNA | 10 ul |
| PCR Water | 1 ul |
| Total | 13 ul |

The annealing mix is then incubated at 65° C. for 5 minutes and then incubated on ice for at least 1 minute.

An enzyme mix is also assembled, as shown in Table 3.

TABLE 3

| Component | Volume |
|---|---|
| 5X SSIV Buffer | 4 ul |
| 10 mM DTT | 1 ul |
| RNaseOUT Recombinant RNase | 1 ul |
| Inhibitor | |
| SuperScript IV Reverse Transcriptase | 1 ul |
| (200 U/ul Pos Control) or PCR Water | |
| Total | 7 ul |

The annealing mix and the enzyme mix are then mixed together and incubated at 50° C. for 10 minutes and with an inactivation step of 80° C. for 10 minutes.

For the amplification step, the following components are assembled as shown in Table 4.

TABLE 4

| Component | Volume | |
|---|---|---|
| NEB Next Q5 2X | 12.5 | ul |
| 10 uM Forward/Reverse Primer ORF5 | 5 | ul |
| RNaseOUT Recombinant RNase | 1 | ul |
| Inhibitor | | |
| cDNA | 2.5 | ul |
| PCR Water | 5 | ul |
| Total | 25 | ul |

Once assembled, the amplification steps are performed as follows: 98° C. for 2 minutes; 35 cycles of 98° C. for 10 s, 55° C.-60° C. for 10 s, 72° C. for 45 s; followed by one cycle at 72° C. for 5 minutes; hold at 4° C.

After the amplification step, the reaction is cleaned up using solid-phase reversible immobilization (SPRI) beads. Beads may be placed in the PCR tube, mixed, and incubated for five minutes at room temperature. The tube may then be placed on a magnetic rack for two minutes, and the supernatant is discarded. The SPRI beads are then washed repeatedly with 80% ethanol. At the final ethanol wash, the tubes are air dried for one minute, and 20 microliters of water are added to the tube, which eluted the DNA from the beads. After 10 minutes at room temperature, the tube is placed on the magnetic rack for two minutes, and the supernatant containing the amplified DNA is removed and placed in a clean tube. The DNA may then be quantified via one or more methods (e.g., Qubit, TapeStation).

### Example 4: Detection of PRRSv from Positive Individuals

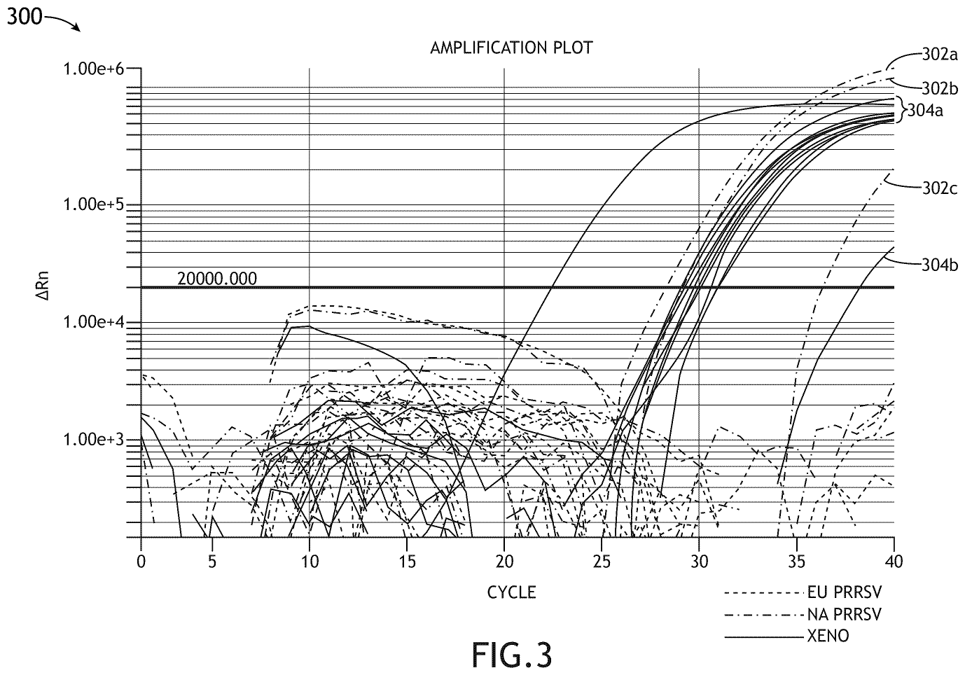

Fecal droppings were collected from individual animals suspected of harboring PRRSv. Portions of the fecal samples were processed to produce 10 ml of slurry from each animal, which were subsequently purified and tested for PRRSv as described herein. Four animals tested positive for PRRSv, as shown in Table 5 and graph 300 of FIG. 3, which illustrates an amplification plot for the biological samples tested for PRRSv. Amplification curves 302a-c for the North American strain of PRRSv “NA PRRSV” are shown, along with amplification curves 304a-c for the positive control “Xeno”. Samples tested for the European strain of PRRSv, “EU PRRSV,” did not produce an amplification curve.

TABLE 5

| Sample number | Sample | PRRSv Ct (NA) |
|---|---|---|
| 1 | Negative sow | Undetermined |
| 2 | PRRSv positive sow 1 | Undetermined |
| 3 | PRRSv positive sow 2 | Undetermined |
| 4 | PRRSv positive sow3 | Undetermined |
| 5 | PRRSv positive sow 4 | Undetermined |
| 6 | PRRSv positive sow 5 | Undetermined |
| 7 | PRRSv positive piglet 1 | 30.733 |
| 8 | PRRSv positive piglet 2 | 29.151 |
| 9 | PRRSv positive piglet 3 | 28.327 |
| 10 | PRRSv positive piglet 4 | 36.396 |
| 11 | PRRSv positive piglet 5 | Undetermined |

“Ct” refers to cycle threshold. Graph 1 shows the four positive samples, along with multiple positive controls (“Xeno”). “NA PRRSv” refers to the North American strain of PRRSv. “EU PRRSv” refers to the European strain of PRRSv.

### Example 5: Detecting PRRSv Using Diluted Slurries Collected from Shoe Covers

Feces samples were collected from a swine facility using the fecal floor method using shoe covers. Fecal slurries were produced from the shoe covers, and viral RNA was extracted from the slurries and tested for PRRSv as described herein. Results of the testing are shown in Table 6.

TABLE 6

| Sample | Target | Ct |
|---|---|---|
| 1 - 1:50 sow shoe cover | Xeno | Undetermined |
| 1 - 1:50 sow shoe cover | EU PRRSv | Undetermined |
| 1 - 1:50 sow shoe cover | NA PRRSv | Undetermined |
| 2 - 1:100 sow shoe cover | Xeno | Undetermined |
| 2 - 1:100 sow shoe cover | EU PRRSv | Undetermined |
| 2 - 1:100 sow shoe cover | NA PRRSv | Undetermined |
| 3 - 1:200 sow shoe cover | Xeno | 31.43 |
| 3 - 1:200 sow shoe cover | EU PRRSv | Undetermined |
| 3 - 1:200 sow shoe cover | NA PRRSv | 34.25 |
| 4 - 1:400 sow shoe cover | Xeno | 31.992 |
| 4 - 1:400 sow shoe cover | EU PRRSv | Undetermined |
| 4 - 1:400 sow shoe cover | NA PRRSv | 36.119 |
| 5 - 1:800 sow shoe cover | Xeno | 32.58 |
| 5 - 1:800 sow shoe cover | EU PRRSv | Undetermined |
| 5 - 1:800 sow shoe cover | NA PRRSv | 37.00 |
| NTC (Negative Control) | Xeno | Undetermined |
| NTC (Negative Control) | EU PRRSv | Undetermined |
| NTC (Negative Control) | NA PRRSv | Undetermined |
| PC (Positive Control) | Xeno | 31.17 |
| PC (Positive Control) | EU PRRSV | Undetermined |
| PC (Positive Control) | NA PRRSV | 31.69 |

Of note, the higher dilutions of slurry (1:200, 1:400, 1:800), were more successful at detecting the North American strain of PRRSv than the lower dilutions (1:50, 1:100). This may indicate that the amplification inhibitors known to exist in the feces of corn-fed animals have been diluted to a concentration where they are unable to inhibit downstream enzymatic reactions.

### Example 6: Side-by-Side Comparison of Fecal Floor Collection Method to the Standard Oral Fluid Collection and Detection Method

A side-by-side comparison of the fecal floor collection and detection method to the standard oral fluid collection and detection method for PRRSv surveillance was performed on Aug. 27, 2024. Samples were collected from rooms 1, 3, and 5 of a swine nursery site in northeast Nebraska, “test site 1,” that had tested negative for oral fluids (within 12 hours of collection by the fecal floor collection and detection method) as performed by Midwest Vet Services. The fecal floor collection and detection method detected PRRSv in all three rooms. Oral fluids were tested again 6 days later by the ISU Veterinary Diagnostic Laboratory (VDL). At this time, samples from rooms 1, 3, and 5 tested positive for PRRSv via the oral fluids testing method. Therefore, it is plausible that the fecal floor collection and detection method detects PRRSv 6 days earlier than the standard oral fluid collection and detection method for PRRSv surveillance. The results of the samples tested via the fecal floor collection and detection method are shown in Table 7.

TABLE 7

| | Sample | | Pos. Con. | PRRSv |
|---|---|---|---|---|
| Room | # | Dilution | “Xeno” | NA Ct |
| 1 | 1 | 100 | 30.44 | 30.22 |
| 1 | 2 | 200 | 27.59 | 30.96 |
| 1 | 3 | 400 | 29.21 | 29.92 |
| 1 | 4 | 800 | 28.94 | 30.03 |
| 3 | 5 | 100 | UND | UND |
| 3 | 6 | 200 | 28.95 | 29.87 |
| 3 | 7 | 400 | 28.37 | 29.31 |
| 3 | 8 | 800 | 27.74 | 29.37 |
| 5 | 9 | 100 | 28.59 | 32.64 |
| 5 | 10 | 200 | 28.76 | 33.54 |
| 5 | 11 | 400 | 28.47 | 32.16 |
| 5 | 12 | 800 | 28.47 | 33.06 |
| Neg. Control | | | UND | UND |
| Pos. Control | | | 29.23 | 28.74 |

“UND” refers to “Undetermined”. Positive results for PRRSv using the oral standard fluid collection method were confirmed by the Iowa State Veterinary Diagnostic Library (VDL).

### Example 7: Daily Comparison of Floor Fecal Testing Methods to Conventional Oral Fluid Testing for PRRSv Detection in Swine Population

In this example, a high-risk commercial nursery and a high-risk commercial finisher facility in northeast Nebraska were selected for evaluation. The nursery contains eight rooms. Each room consists of 24 pens, which will be stocked at around 3 sq. feet. The estimated average site inventory is 10,500 pigs. Pigs are placed at 21 days of age and remain in the nursery for 7 weeks; at which time they are transported to a finisher. The finisher setup may vary, with anticipated test sites having ten rooms with four pens per room, stocked at 7.2 sq. feet. The average site inventory at the finisher is approximately 14,000 pigs.

Pigs are placed at 10 weeks of age and are marketed at 30 weeks of age. Pigs at all locations are PRRS naïve, and none are PRRS vaccinated. Sampling will occur weekly, beginning the week after placement until the time of shipment or until a PRRS introduction at the site. Once PRRS is detected, then daily room testing follows. Sampling will occur at the room level.

Two methods were compared in this study. First, a traditional oral fluid sampling method (e.g., the control method) was used using a standard operating procedure, where six collection strands were placed spatially throughout the room and pooled into one tube at the time of collection. The samples were processed via RT-qPCR to determine PRRSv presence and Ct values. Second, the floor feces sampling method (e.g., the method of the current disclosure) was used using the protocols as described herein. For example, samples were collected using shoe covers, and the PRRSv detection protocols were performed as described.

In the Finisher, room 10 tested positive for PRRSv on day one of surveillance using the fecal floor collection protocol, whereas the oral fluids collection protocol first detected PRRSv on day five, as shown in Table 8. Pens where oral fluids tested positive for PRRSv were no longer assayed for PRRSv by the oral fluids method.

TABLE 8

| Date Sample | | | | Days |
|---|---|---|---|---|
| Collected | Room | Fecal Floor Ct | Oral Fluids Ct | After |
| Mar. 20, 2025 | Finisher | 35.589/POSITIVE | NEG | 0 |
| | Rm 10 | | | |
| Mar. 21, 2025 | Finisher | 34.533/POSITIVE | NEG | 1 |
| | Rm 10 | | | |
| Mar. 22, 2025 | Finisher | 29.9/POSITIVE | NEG | 2 |
| | Rm 10 | | | |
| Mar. 23, 2025 | Finisher | 33.74/POSITIVE | NEG | 3 |
| | Rm 10 | | | |
| Mar. 24, 2025 | Finisher | 35.54/POSITIVE | 33.90 POSITIVE | 4 |
| | Rm 10 | | | |
| Mar. 25, 2025 | Finisher | 33.8/POSITIVE | 31.00 POSITIVE | n/a |
| | Rm 10 | | | |
| Mar. 26, 2025 | Finisher | 33.18/POSITIVE | n/a | n/a |
| | Rm 10 | | | |

In the nursery facility, room one tested positive for PRRSv on day two of surveillance using the Fecal Floor collection protocol, whereas the oral fluids collection protocol first detected PRRSv on day eight, as shown in Table 9.

TABLE 9

| Date | | | | |
|---|---|---|---|---|
| Sample | | Fecal | Oral | Days |
| Collected | Room | Floor Ct | Fluids Ct | After |
| Apr. 2, 2025 | Nursery Room 1 | UND | NEG | n/a |
| Apr. 3, 2025 | Nursery Room 1 | 29.136/ | NEG | 0 |
| | | POSITIVE | | |
| Apr. 4, 2025 | Nursery Room 1 | UND | NEG | 1 |
| Apr. 5, 2025 | Nursery Room 1 | 34.727/ | NEG | 2 |
| | | POSITIVE | | |
| Apr. 6, 2025 | Nursery Room 1 | UND | NEG | 3 |
| Apr. 7, 2025 | Nursery Room 1 | 34.408/ | NEG | 4 |
| | | POSITIVE | | |
| Apr. 8, 2025 | Nursery Room 1 | UND | NEG | 5 |
| Apr. 9, 2025 | Nursery Room 1 | 34.171/ | 34.4/ | 6 |
| | | POSITIVE | POSITIVE | |
| Apr. 10, 2025 | Nursery Room 1 | UND | | |

In the nursery facility, room two tested positive for PRRSv on day three of surveillance using the Fecal Floor collection protocol, whereas the oral fluids collection protocol first detected PRRSv on day eight, as shown in Table 10.

TABLE 10

| Date | | | | |
|---|---|---|---|---|
| Sample | | Fecal | Oral | Days |
| Collected | Room | Floor Ct | Fluids Ct | After |
| Apr. 2, 2025 | Nursery Room 2 | UND | NEG | n/a |
| Apr. 3, 2025 | Nursery Room 2 | UND | NEG | n/a |
| Apr. 4, 2025 | Nursery Room 2 | 37.363/ | NEG | 0 |
| | | POSITIVE | | |
| Apr. 5, 2025 | Nursery Room 2 | UND | NEG | 1 |
| Apr. 6, 2025 | Nursery Room 2 | UND | NEG | 2 |
| Apr. 7, 2025 | Nursery Room 2 | 35.387/ | NEG | 3 |
| | | POSITIVE | | |
| Apr. 8, 2025 | Nursery Room 2 | 33.236/ | NEG | 4 |
| | | POSITIVE | | |
| Apr. 9, 2025 | Nursery Room 2 | 30.814/ | 35.5/ | 5 |
| | | POSITIVE | POSITIVE | |
| Apr. 10, 2025 | Nursery Room 2 | 28.617/ | | |
| | | POSITIVE | | |

In the Nursery facility, room four tested positive for PRRSv on day two of surveillance using the Fecal Floor collection protocol, whereas the oral fluids collection protocol first detected PRRSv on day four, as shown in Table 11.

TABLE 11

| Date | | | | |
|---|---|---|---|---|
| Sample | | Fecal | Oral | Days |
| Collected | Room | Floor Ct | Fluids Ct | After |
| Apr. 2, 2025 | Nursery Room 4 | UND | NEG | n/a |
| Apr. 3, 2025 | Nursery Room 4 | 32.565/ | NEG | 0 |
| | | POSITIVE | | |
| Apr. 4, 2025 | Nursery Room 4 | UND | NEG | 1 |
| Apr. 5, 2025 | Nursery Room 4 | 33.537/ | 36.9/ | 2 |
| | | POSITIVE | POSITIVE | |
| Apr. 6, 2025 | Nursery Room 4 | UND | NEG | X |
| Apr. 7, 2025 | Nursery Room 4 | 31.611/ | 34.4/ | X |
| | | POSITIVE | POSITIVE | |
| Apr. 8, 2025 | Nursery Room 4 | 32.967/ | 34.7/ | X |
| | | POSITIVE | POSITIVE | |

In the finisher facility, room six tested positive for PRRSv on day three of surveillance using the Fecal Floor collection protocol, whereas the oral fluids collection protocol first detected PRRSv on day eight, as shown in Table 12.

TABLE 12

| Date | | | | |
|---|---|---|---|---|
| Sample | | Fecal | Oral | Days |
| Collected | Room | Floor Ct | Fluids Ct | After |
| Apr. 2, 2025 | Nursery Room 6 | 29.54/ | NEG | 0 |
| | | POSITIVE | | |
| Apr. 3, 2025 | Nursery Room 6 | 34.073/ | NEG | 1 |
| | | POSITIVE | | |
| Apr. 4, 2025 | Nursery Room 6 | 32.202/ | NEG | 2 |
| | | POSITIVE | | |
| Apr. 5, 2025 | Nursery Room 6 | 33.854/ | 32.9/ | 3 |
| | | POSITIVE | POSITIVE | |
| Apr. 6, 2025 | Nursery Room 6 | 34.283/ | 31.3/ | X |
| | | POSITIVE | POSITIVE | |
| Apr. 7, 2025 | Nursery Room 6 | 31.848/ | 30.6/ | X |
| | | POSITIVE | POSITIVE | |
| Apr. 8, 2025 | Nursery Room 6 | 28.519/ | 30.9/ | X |
| | | POSITIVE | POSITIVE | |

In the finisher facility, room seven tested positive for PRRSv on day two of surveillance using the fecal floor collection protocol, whereas the oral fluids collection protocol first detected PRRSv on day seven, as shown in Table 13.

TABLE 13

| Date | | | | |
|---|---|---|---|---|
| Sample | | Fecal | Oral | Days |
| Collected | Room | Floor Ct | Fluids Ct | After |
| Apr. 2, 2025 | Nursery Room 7 | UND | NEG | n/a |
| Apr. 3, 2025 | Nursery Room 7 | 38.761/ | NEG | 0 |
| | | POSITIVE | | |
| Apr. 4, 2025 | Nursery Room 7 | UND | NEG | 1 |
| Apr. 5, 2025 | Nursery Room 7 | UND | NEG | 2 |
| Apr. 6, 2025 | Nursery Room 7 | UND | NEG | 3 |
| Apr. 7, 2025 | Nursery Room 7 | UND | NEG | 4 |
| Apr. 8, 2025 | Nursery Room 7 | UND | 35.5/ | 5 |
| | | | POSITIVE | |
| Apr. 9, 2025 | Nursery Room 7 | 33.585/ | 32.8/ | X |
| | | POSITIVE | POSITIVE | |
| Apr. 10, 2025 | Nursery Room 7 | 31.308/ | X | X |
| | | POSITIVE | | |

The results shown in tables 8-13 show that the fecal floor method for detection of PRRSv within a population of animals is capable of detecting PRRSv at an earlier time point than the oral fluids method. For example, Finisher Room 10 tested positive for PRRSv on the first day of testing, via the floor fecal method, whereas the oral fluids method did not detect PRRSv until four days later (e.g., as shown in Table 7). In another example, Nursery Room 1 tested positive for PRRSv on the second day of testing, via the floor fecal method, whereas the oral fluids method did not detect PRRSv until six days later (e.g., as shown in Table 8). The ability of the fecal floor method to detect pathogen nucleic acids days before detection by the “standard” oral fluids method may be due to one or more of several possible explanations, including, but not limited to, higher pathogen count in feces per animal, and greater collections of biological material per animal, and higher number of animals screened per sample.

### Example 8: Daily Comparison of Floor Fecal Testing Methods to Conventional Oral Fluid Testing for PRRSv Detection in Swine Population

In another comparison between the oral fluids surveillance method and the current fecal floor method, another nursery similar to that tested above was evaluated for the presence of PRRSv. Samples were collected daily for both the current fecal floor method and oral fluids, with collections continuing until all rooms were positive for both methods. All fecal floor samples from each barn room were made into 1:200 and 1:600 slurry prior to enrichment step. Ct values for collected sample are reported below in Table 14.

TABLE 14

| Date | | | | |
|---|---|---|---|---|
| Sample | | Fecal Floor | Oral Fluids | Days |
| Collected | Room | Ct | Ct | After |
| May 27, 2025 | 20 | UND | NEG | |
| May 29, 2025 | 20 | 33.66 | NEG | 0 |
| May 30, 2025 | 20 | UND | NEG | 1 |
| May 31, 2025 | 20 | UND | NEG | 2 |
| Jun. 1, 2025 | 20 | 35.39 | NEG | 3 |
| Jun. 2, 2025 | 20 | 34.75 | NEG | 4 |
| Jun. 3, 2025 | 20 | 34.18 | 35.00 | 5 |
| Jun. 4, 2025 | 20 | 34.37 | Collected | |
| Jun. 5, 2025 | 20 | 32.42 | Collected | |
| May 27, 2025 | 21 | UND | NEG | |
| May 29, 2025 | 21 | UND | NEG | |
| May 30, 2025 | 21 | 33.85 | NEG | 0 |
| May 31, 2025 | 21 | UND | NEG | 1 |
| Jun. 1, 2025 | 21 | UND | NEG | 2 |
| Jun. 2, 2025 | 21 | 34.57 | NEG | 3 |
| Jun. 3, 2025 | 21 | 33.05 | 33.1 | 4 |
| Jun. 4, 2025 | 21 | 31.36 | Collected | |
| Jun. 5, 2025 | 21 | 28.28 | Collected | |
| May 27, 2025 | 22 | 30.16 | Positive | |
| May 29, 2025 | 22 | 27.85 | 30.5 | |
| May 30, 2025 | 22 | 26.54 | 28.5 | |
| May 31, 2025 | 22 | 24.77 | 28.2 | n/a |
| May 27, 2025 | 23 | UND | NEG | |
| May 29, 2025 | 23 | UND | NEG | |
| May 30, 2025 | 23 | 36.11 | NEG | 0 |
| May 31, 2025 | 23 | 33.15 | NEG | 1 |
| Jun. 1, 2025 | 23 | 31.36 | 33.5 | 2 |
| Jun. 2, 2025 | 23 | 32.78 | 32.4 | |
| Jun. 3, 2025 | 23 | 31.78 | 30.7 | |
| Jun. 4, 2025 | 23 | 27.53 | Collected | |
| May 27, 2025 | 24 | UND | NEG | |
| May 29, 2025 | 24 | UND | NEG | |
| May 30, 2025 | 24 | 35.18 | NEG | 0 |
| May 31, 2025 | 24 | 30.24 | NEG | 2 |
| Jun. 1, 2025 | 24 | 30.23 | NEG | 3 |
| Jun. 2, 2025 | 24 | 35.51 | 34.7 | 4 |
| Jun. 3, 2025 | 24 | 32.12 | 32.3 | |
| Jun. 4, 2025 | 24 | 27.31 | Collected | |
| Jun. 5, 2025 | 24 | 28.47 | Collected | |

The results in Table 14 show that the fecal floor method for detection of PRRSv within a population of animals is capable of detecting PRRSv at an earlier time point than the oral fluids method. Importantly, in rooms 20 to 24, the fecal floor method detected PRRSv an average of 3.75 days before detection by the oral fluids method.

### Example 9: Detection of PRRSv Via Nanopore Sequencing

Fecal floor samples testing positive for PRRSv were reanalyzed, this time for sequencing via a MiniON™ flow cell, available from Oxford Nanopore Technologies. Nucleic Acids were isolated from the samples as described herein, and reverse transcription and PCR reactions were performed. For these samples, random hexamers were used in the reverse transcription step, along with a 10-minute preincubation step at 23° C. The number of thermocycle cycles were also increased from 30 to 35 cycles. After a PCR and PCR-cleanup, samples were analyzed via the flow cell, of which several amplicons were identified. Blast searching of the amplicons through the ISU PRRSView BLAST Tool revealed that the amplicon sequences matched recent PRRSv sequences added to the PRRSVIEW database to over 99.6 percent, as reported in Table 15.

TABLE 15

| PRRSVIEW | Received | | | | | Query |
|---|---|---|---|---|---|---|
| Sequence | Date | State | Country | Lineage | Variant | Identity |
| 1 | 2025 May | NE | USA | L1C.5 | 1C.5.33 | 99.669% |
| | 28 | | | | | |
| 2 | 2025 Jun. | NE | USA | L1C.5 | 1C.5.33 | 99.669% |
| | 5 | | | | | |

In embodiments, the fecal floor method detects pathogenic nucleic acids (e.g., nucleic acids for PRRSv) more than one day earlier than another surveillance method (e.g., the oral fluids method). For example, the fecal floor method may detect pathogenic nucleic acids more than two days earlier than another surveillance method. In another example, the fecal floor method may detect pathogenic nucleic acids four or more days earlier than another surveillance method. In another example, the fecal floor method may detect pathogenic nucleic acids six or more days earlier than another surveillance method. In another example, the fecal floor method may detect pathogenic nucleic acids eight or more days earlier than another surveillance method.

In a prophetic embodiment, the fecal floor method detects pathogenic nucleic acids other than those for PRRSv more than one day earlier than another surveillance method (e.g., the oral fluids method). These pathogenic nucleic acids may include nucleic acids for swine influenza virus, PEDv, porcine deltacoronavirus (PDCOV), TGEv, and Mycoplasma hyopneumoniae. For example, the fecal floor method may detect nucleic acids for one of these pathogens four or more days earlier than another surveillance method. In another example, the fecal floor method may detect nucleic acids for one of these pathogens six or more days earlier than another surveillance method.

In embodiments, the surveillance method described herein may be used to detect a small number of positive-testing animals within a population of animals. For example, the method may be configured to detect a single positive-testing animal from a sample containing feces from a population of 100 or more animals. In another example, the method may be configured to detect a single positive-testing animal from a sample containing feces from a population of 1000 or more animals.

The oral fluids collection protocol may include one or more steps as described herein. For example, the oral fluids collection protocol may include suspending a length of cotton rope in a location accessible to the pigs. Ropes should be placed in a clean area of the pen and not in close proximity to water or feed. They can be placed on gates between two pens to increase sampling numbers per rope and increase spatial sampling area. Cotton rope is recommended because it is highly absorbent. Rope that is ½″ (1.3 cm) thick may be used for nursery pigs while ⅝″ (1.6 cm) rope may be used for grow-finish pigs. As an example, for an oral fluids protocol performed on a barn that includes approximately 1400 pigs per barn dispersed across 25 pens, six oral fluid ropes were hung across 12 of the pens.

The oral fluids collection protocol may further include hanging the rope shoulder-high to the pigs. In active pens, 20-30 minutes is sufficient sampling time. For younger pigs, you may need to hang for closer to 1 hour or train them the day before. The oral fluids collection protocol may further include extracting oral fluids from the rope. For example, the bottom (wet) end of the rope may be inserted into a clean plastic bag and fluid wrung out so that the fluid accumulates in one corner. The collected sample may then be analyzed (e.g., via qPCR).

While implementations of method 100 are discussed herein, it is further contemplated that various steps of method 100 may be included, excluded, rearranged, and/or implemented in many ways without departing from the essence of the present disclosure. Accordingly, the foregoing embodiments and implementations of method 100 are included by way of example only and are not intended to limit the present disclosure in any way. It is further contemplated that each of the embodiments of the method described above may include any other step(s) of any other method(s) described herein.

In embodiments, the systems and methods herein may include the use of controls as needed to perform the method, such as the internal RNA control. For example, the method may include, but not be limited to, a starting enrichment known positive control, a known negative slurry control, a positive PCR PRRSv control, a water (e.g., negative) PCR control. For instance, the method may include the use of a Bovine Coronavirus control as an enrichment internal control to assure the trapping beads are trapping pathogens at the expected efficiency.

Although particular embodiments of this invention have been illustrated, it is apparent that various modifications and embodiments of the invention may be made by those skilled in the art without departing from the scope and spirit of the foregoing disclosure. It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Accordingly, the scope of the invention should be limited only by the claims appended hereto.

As used herein, a “sample” refers to any substance containing or presumed to contain nucleic acids and includes a sample of tissue or fluid isolated from an individual or individuals. Particularly, the nucleic acid sample may be obtained from a single cell, an organism or a combination of organisms selected from viruses, bacteria, fungi, plants, and animals. Preferably, the nucleic acid sample is obtained from a mammal. In a preferred embodiment, the mammal is human. The nucleic acid sample can be obtained from a specimen of body fluid or tissue biopsy of a subject, or from cultured cells. The body fluid may be selected from whole blood, serum, plasma, urine, sputum, bile, stool, bone marrow, lymph, semen, breast exudate, bile, saliva, tears, bronchial washings, gastric washings, spinal fluids, synovial fluids, peritoneal fluids, pleural effusions, and amniotic fluid.

As used herein, the term “nucleotide” generally refers to the monomer components of nucleotide sequences even though the monomers may be nucleoside and/or nucleotide analogs, and/or modified nucleosides such as amino modified nucleosides in addition to nucleotides. In addition, “nucleotide” includes non-naturally occurring analog structures. Nucleotide may be deoxyribonucleotides, ribonucleotides, or other nucleic acids. As used herein, the term “nucleotide sequence” refers to either a homopolymer or a heteropolymer of deoxyribonucleotides, ribonucleotides, or other nucleic acids.

As used herein, the term “nucleic acid” refers to at least two nucleotides covalently linked together. A nucleic acid will generally contain phosphodiester bonds, although in some cases nucleic acid analogs are included that may have alternate backbones. Nucleic acids may be single-stranded or double-stranded, as specified, or contain portions of both double-stranded and single-stranded sequence. The nucleic acid may be DNA, both genomic and cDNA, RNA, DNA and RNA mixtures, or DNA-RNA hybrids, where the nucleic acid contains any combination of deoxyribo- and ribo-nucleotides, and any combination of bases, including uracil, adenine, thymine, cytosine, guanine, inosine, xathanine, hypoxathanine, etc. Reference to a “DNA sequence” or “RNA sequence” can include both single-stranded and double-stranded DNA or RNA. A specific sequence, unless the context indicates otherwise, refers to the single stranded DNA or RNA of such sequence, the duplex of such sequence with its complement (double stranded DNA or RNA) and/or the complement of such sequence.

As used herein, the “polynucleotide” and “oligonucleotide” are types of “nucleic acid”, and generally refer to primers, or oligomer fragments to be detected. There is no intended distinction in length between the term “nucleic acid”, “polynucleotide”, and “oligonucleotide”, and these terms will be used interchangeably. “Nucleic acid”, “DNA”, and “RNA” and similar terms also include nucleic acid analogs. The oligonucleotide is not necessarily physically derived from any existing or natural sequence but may be generated in any manner, including chemical synthesis, DNA replication, reverse transcription, or a combination thereof.

As used herein, the terms “target sequence”, “target nucleic acid”, “target polynucleotide”, and “nucleic acids of interest” are used interchangeably and refer to a desired region which is to be either amplified, detected, or both, or is the subject of hybridization with a complementary oligonucleotide, polynucleotide, e.g., a blocking oligomer, or the subject of a primer extension process. The target sequence can be composed of DNA, RNA, analogs thereof, or combinations thereof. The target sequence can be single-stranded or double-stranded. In extension processes, the target polynucleotide which forms a hybridization duplex with an oligonucleotide (template) may be referred to as a “primer”, or the target nucleic acid which forms a hybridization duplex with the primer may also be referred to as a “template.” A template serves as a pattern for the synthesis of a complementary polynucleotide. A target sequence may be derived from any living or once living organism, including but not limited to prokaryotes, eukaryotes, plants, animals, and viruses, as well as synthetic and/or recombinant target sequences, or a combination thereof.

“Primer” as used herein refers to an oligonucleotide or polynucleotide, whether occurring naturally or produced synthetically, which is capable of acting as a point of initiation of synthesis when placed under conditions in which synthesis of a primer extension product, which is complementary to a nucleic acid strand is induced i.e., in the presence of nucleotides and an agent for polymerization and at a suitable temperature and in a suitable buffer. Such conditions include the presence of four or more different deoxyribonucleoside triphosphates and a polymerization-inducing agent such as DNA polymerase, and/or RNA polymerase, and/or reverse transcriptase, in a suitable buffer (“buffer” includes substituents which are cofactors, or affect pH, ionic strength, etc.), and at a suitable temperature. The primers herein are selected to be substantially complementary to a strand of each specific sequence to be extended. This means that the primers must be sufficiently complementary to hybridize with their respective strands. A non-complementary nucleotide may be present.

As used herein, “pooled sample” refers to the collection of a sample than includes biological material from more than one animal. For example, when using shoe covers to collect biological samples off of a facility floor, the shoe covers are effectively collecting pooled samples, as the feces on the floor of the facility typically belong to several, if not hundreds of individual animals. Once the biological sample is extracted from the shoe cover, the biological sample remains as a pooled sample. Biological samples from multiple shoe covers may also be pooled together, adding to the diversity of the pooled sample.

As used herein, “amplification” refers to the process of semi-conservatively replicating nucleic acid strands by enzyme-catalyzed extension. Exemplary enzymes for amplification of nucleic acids in the present disclosure include, for example, nucleic acid polymerases. In some embodiments, an isothermal polymerase is used to amplify nucleic acids. In some embodiments, amplification is carried out with a high-fidelity polymerase with the technique known as the polymerase chain reaction (PCR). Amplification can be performed with natural and nonnatural nucleotide bases, ribonucleotide bases or deoxyribonucleotide bases, labeled nucleotide bases, and the like.

As used herein “reverse transcription-polymerase chain reaction” or “RT-PCR” refers to the well-known technique for amplification and detection of a target RNA sequence wherein an RNA-dependent DNA polymerase having reverse transcriptase (RT) activity is used to make a complementary DNA (cDNA) from an RNA target sequence, and the cDNA is then amplified by PCR to produce multiple copies of DNA.

As detailed herein, obtaining a sample and performing PCR may also include harvesting the pathogen and isolating pathogen DNA from the harvested pathogens for amplification by PCR. Harvesting the pathogen may be performed by methods (e.g., centrifugation, kits) that are well-known to the skilled artisan. Isolating pathogen DNA from the harvested pathogens may performed by methods (e.g., phenol extraction/kits) that are well-known to the skilled artisan.

As used herein, the term “well” refers to a single container or reaction vessel. Though the term well is often used when referring to plates or microplates, it is to be understood that the methods of the present disclosure may also be performed using, for example, tubes or other vessels capable of containing and separating liquids.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having six members refers to groups having 1, 2, 3, 4, or 6 members, and so forth.

One skilled in the art will recognize that the herein described components operations, devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components, operations, devices, and objects should not be taken as limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” and the like). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, and the like” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, and the like). In those instances where a convention analogous to “at least one of A, B, or C, and the like” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, and the like). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

Finally, as used herein any reference to “in embodiments, “one embodiment”, “some embodiments”, or the like means that a particular element, feature, structure, or characteristic described in connection with the embodiment is included in at least one embodiment disclosed herein. The appearances of the phrase “in embodiments” in various places in the specification are not necessarily all referring to the same embodiment, and embodiments may include one or more of the features expressly described or inherently present herein, or any combination of sub-combination of two or more such features, along with any other features which may not necessarily be expressly described or inherently present in the instant disclosure.

What is claimed:

1. A method for surveilling a population of animals within a facility for one or more pathogens comprising:
sampling feces from a floor of the facility;
isolating the one or more pathogens or nucleic acids from the one or more pathogens from a sampled feces;
detecting the one or more pathogens from the sampled feces based on the nucleic acids from the one or more pathogens from the sampled feces; and
reporting a detection of the one or more pathogens.

2. The method of claim 1, wherein sampling feces from the floor of the facility comprises:
walking along the floor within the facility with footwear, wherein the footwear comprises an absorbent cover;
absorbing feces from the floor onto the absorbent cover;
removing the footwear; and
transferring the feces from the absorbent cover to a sample container.

3. The method of claim 2, wherein transferring the feces from the absorbent cover to the sample container comprises:
placing the absorbent cover in an immersion container, wherein the footwear comprises a shoe cover;
adding a diluent to the immersion container, wherein adding the diluent transfers the feces from the absorbent cover and produces a slurry produces a slurry; and
transferring the slurry from the immersion container to the sample container.

4. The method of claim 3, wherein isolating the one or more pathogens from the sampled feces comprises:
adding trapping particles to the slurry, wherein the trapping particles bind the one or more pathogens or the nucleic acids from the one or more pathogens;
washing the trapping particles;
adding a lysis buffer; wherein the lysis buffer causes a lysis of pathogens bound by the trapping particles;
heating the lysis buffer and the trapping particles for at 90° C. or greater for more than one minute;
removing a supernatant from the trapping particles, wherein the supernatant contains the nucleic acids from the one or more pathogens;
adding nucleic acid binding substrate to the supernatant wherein the supernatant contains the nucleic acids from the one or more pathogens;
washing the nucleic acid binding substrate; and
eluting the nucleic acids from the nucleic acid binding substrate into an elution sample.

5. The method of claim 4, further comprising before washing the nucleic acid binding substrate, adding proteinase K.

6. The method of claim 4, wherein detecting the one or more pathogens from the sampled feces comprises:
obtaining a portion of the elution sample;
reverse transcribing RNA from the portion of the elution sample, producing complementary cDNA;
amplifying a region of a genome or mRNA of the one or more pathogens from the complementary cDNA, producing an amplified product; and
detecting the amplified product.

7. The method of claim 6, wherein the amplified product comprises a portion of a porcine reproductive and respiratory syndrome virus (PRRSv) genome.

8. The method of claim 7, wherein the amplified product comprises open reading frame five (ORF5) of the PRRSv genome.

9. The method of claim 7, wherein the method detects PRRSv in the population of animals four days before a detection by an oral fluids detection protocol.

10. The method of claim 6, wherein the slurry comprises feces from more than 100 animals of the population of animals, wherein the method is configured to detect a single positive-testing animal within the slurry.

11. The method of claim 6, wherein the amplified product comprises a portion of swine influenza virus.

12. The method of claim 6, wherein the amplified product comprises a portion of porcine deltacoronavirus (PDCoV) genome.

13. The method of claim 6, wherein the amplified product comprises a portion of porcine epidemic diarrhea Virus (PEDv) genome.

14. The method of claim 6, wherein the amplified product comprises a portion of transmissible gastroenteritis virus (TGEV) genome.

15. The method of claim 4, wherein detecting the one or more pathogens from the sampled feces comprises:
obtaining a portion of the elution sample;
amplifying a region of a genome or mRNA of the one or more pathogens from the elution sample, producing an amplified product; and
detecting the amplified product.

16. The method of claim 4, further comprising before washing the nucleic acid binding substrate, adding a control nucleic acid, wherein the control nucleic acid is eluted in the elution sample, amplified, and detected.

17. The method of claim 4, wherein one or more of the nucleic acids from the one or more pathogens are detected via nanopore sequencing.

18. The method of claim 1, wherein reporting the detection of the one or more pathogens comprises:
providing a forecast of a disease state for a population of animals for one or more of the one or more pathogens.

19. The method of claim 1, wherein reporting the detection of the one or more pathogens comprises:
obtaining a trained artificial intelligence (AI) and/or machine learning (ML) model; and
based at least on the detecting of the one or more pathogens from the sampled feces and the trained AI and/or ML model, inferring a forecast of a disease state for the population of animals.

20. A kit for surveilling a population of animals within a facility for one or more pathogens comprising:
a shoe cover configured to absorb feces;
an immersion container configured to receive the shoe cover and a diluent, wherein the shoe cover has absorbed feces, wherein the immersion container is configured to contain a slurry formed from an addition of the diluent to the shoe cover; and
a collection container configured to receive the slurry from the immersion container.
